(19) United States
(12) Plant Patent Application Publication
(10) Pub. No.: US 2010/0251444 P1
(43) Pub. Date: Sep. 30, 2010

(54) CALADIUM PLANT NAMED 'UF404' TITLE AMENDED BY EXAMINER /SBME/ 6-20-10

(50) ;

(75) Inventors: Zhanao Deng, Ellenton, FL (US); Brent Harbaugh, Bradenton, FL (US)

Correspondence Address: University of Florida - ATTN: John Beuttenmuller, PO Box 110200, Gainesville, FL, 32611-0200, US

(73) Assignee: University of Florida Board of Trustees

(21) Appl. No.: 12/383,383

(22) Filed: Mar. 24, 2009

(51) Int. Cl. A01H 5/00

(52) U.S. Cl. PLT/263.1

(57) ABSTRACT

A new Caladium plant named ‘UF404’ particularly distinguished by its bright red lance leaves with white blotches scattered between main veins, and demonstrated potential to produce attractive and sun-tolerant plants in outdoor sunny landscapes, and produce attractive pot plants when tubers are forced in containers, and with improved resistance to chilling injuries, is disclosed.

### ACKNOWLEDGEMENT OF FEDERAL RESEARCH SUPPORT

This invention was made with government support under FLA-BRA-04162 awarded by the Cooperative State Research, Education, and Extension Service, USDA. The government has certain rights in the invention.

### GENUS AND SPECIES

Caladium×hortulanum

### VARIETY DENOMINATION

‘UF404’

### BACKGROUND OF THE NEW PLANT

The invention relates to a new and distinct variety of Caladium×hortulanum plant named ‘UF404’. ‘UF404’ originated from a controlled pollination made in 2003 between ‘Florida Blizzard’ (U.S. Plant Pat. No. 14,406) and ‘Florida Red Ruffles’ (U.S. Plant Pat. No. 13,136). ‘Florida Blizzard’ was selected as the seed parent because of its plant vigor, unique leaf coloration pattern, and high tuber yield. ‘Florida Red Ruffles’ was selected as the pollen parent because of its bright color, compact growth habit, excellent sun burn tolerance, and chilling resistance. ‘Florida Blizzard’ was developed from a cross between ‘Aaron’ (unpatented) and ‘White Christmas’ (unpatented). ‘Florida Red Ruffles’ was developed from a cross between ‘Red Frill’ (unpatented) and ‘Candidum Junior’ (unpatented). The ancestry of ‘Aaron’, ‘Candidum Junior’, ‘Red Frill’, and ‘White Christmas’ is unknown, although ‘Candidum Junior’ was suspected to be a field mutation of ‘Candidum’ (unpatented). Asexual propagation of tubers and evaluation in field and pot studies in Wimauma, Fla. since 2004 have shown that the unique features of ‘UF404’ are stable and reproduced true to type in successive generations of asexual propagation.

Plant Breeder's Rights for this cultivar have not been applied for. ‘UF404’ has not been made publicly available more than one year prior to the filing of this application.

### SUMMARY OF THE INVENTION

Caladium [Caladium×hortulanum Birdsey, Araceae Juss.] is commonly used as a pot or landscape plant and is valued for its colorful leaves. Commercial caladium plants are grown from tubers. The commercial value of a caladium cultivar depends on its tuber yield, leaf color, performance in the landscape as a garden plant, and performance in containers as a pot plant.

The new and distinct variety of caladium is an attractive red lance-leaved caladium that possess a novel combination of foliar characteristics. ‘UF404’ is distinct from other lance-leaved varieties in that it has bright red leaves with white blotches scattered between main veins. ‘UF404’ has been comparable or superior to ‘Florida Red Ruffles’ in tuber yield, pot plant quality, and landscape performance. ‘UF404’ develops numerous brightly colored, wide, and lance-shaped leaves that are resistant to sun burn and the new variety is well suited for forcing in various sized containers, for producing hanging baskets, and for planting in sunny or shady landscape locations. Tubers of ‘UF404’ are well branched and do not require de-eyeing for forcing in small or medium-sized pots (11.4 to 15.3 cm) (FIG. 1), and its leaves are more resistant to chilling injuries than leaves of ‘Florida Red Ruffles’.

### DESCRIPTION OF THE PHOTOGRAPHS

This new caladium plant is illustrated by the accompanying photograph which shows the plant's form and foliage. The new caladium has not been observed under all possible environmental conditions. Its phenotype may vary somewhat with variations in the environment such as light intensity and temperature, without, however, any variance in genotype. The colors shown are as true as can be reasonably obtained by conventional photographic procedures. Colors in the photograph may differ slightly from the color values cited in the detailed botanical description, which accurately describe the colors of the new Caladium.

FIG. 1 shows the overall plant appearance and habit including foliage from a side perspective view of a typical plant of the new Caladium grown in a container.

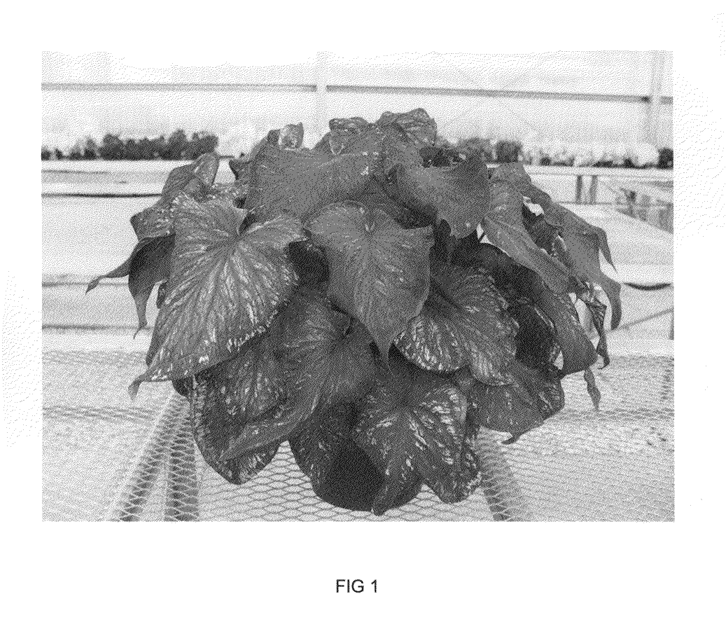

### DESCRIPTION OF THE NEW CULTIVAR

The following detailed description sets forth the distinctive characteristics of ‘UF404’ with color terminology in accordance with British Color Council and The Royal Horticultural Society, Horticultural Colour Chart, except where general color terms of ordinary dictionary significance are obvious. Wherein dimensions, sizes, and other characteristics are given, it is to be understood that such characteristics are approximations of averages set forth as accurately as practicable. The description herein is from 4 month-old specimens grown in Wimauma, Fla., in 2007. Plants used for describing color were grown in 20.3-cm containers in a 45% shaded greenhouse from four No. 1 (3.8 to 6.4 cm in diameter) tubers.

### DETAILED BOTANICAL DESCRIPTION

Classification:
   Botanical.—Caladium×hortulanum.
   Common name.—Caladium.
Parentage:
   Female parent.—‘Florida Blizzard’ (U.S. Plant Pat. No. 14,406) caladium.
   Male parent.—‘Florida Red Ruffles’ (U.S. Plant Pat. No. 13,136) caladium.
Propagation:
   Type.—By tuber division.
   Time to develop roots and sprout.—30 days (Spring — 15° C. night to 29° C. day). 16 days (Summer — 21° C. night to 35° C. day).
   Root description.—Dense, moderately thick roots (up to 2.5 mm at the basal end) with little branching and few lateral roots.
Plant description:
   Plant shape.—Mounding, symmetrical.
   Plant height.—About 27 cm from top of soil to top of leaf plane 4 months from planting tubers in ground beds in full sun.
   Plant width.—About 30-50 cm 4 months from planting tubers in ground beds in full sun.
   Leaf blade.—Sagitate-cordate, up to 25 cm long and 18 cm wide, and slightly undulate, with two lobes 1/7 to ⅛ of the leaf length. The upper surface has dark green (RHS 136B) margins, up to 10 mm wide, bordering the entire leaf except for the basal leaf valley where it is greyed-green (RHS 194B). The central and main veins are greyed-purple (RHS 185B) and often connect marginally with a thin (<1 mm wide) greyed-purple vein (RHS 184A) that parallels with the leaf margin. Interveinal areas are variable in color, grey-purple (RHS 184C) near the leaf center to yellow-green (RHS 147), greyed-orange (RHS 166A) toward the margin and have irregular blotches variable in color, from yellow-white (RHS 158C), to red (RHS 54D) or red-purple (RHS 65D), occurring on up to 85% of the leaf blade and marginal area. The undersurface has a yellow-green (RHS 147B) margin, up to 15 mm wide, and red-purple (RHS 59D) veins. The central and main veins are covered with greyed-white (RHS 155B) wax. Interveinal areas are variable in color and have irregular blotches, variable in color, occurring on up to 85% of the center and marginal area.
   Petiole.—Aspect: Mostly erect, curving outwardly with development. Length: 25-35 cm. Diameter (distal): 5.5 mm. Diameter (proximal): 8.5 mm. Strength: Strong, flexible. Color (distal): Greyed-Purple (RHS 185A). Color (proximal): Greyed-Purple (RHS 187A). Wing length: 2.5-4.5 cm. Wing diameter: 6-9 mm. Wing color: Black (RHS 202A) with streaks of orange-white (RHS 159B).
   Tuber.—Jumbo-sized (6.4 to 8.9 cm in diameter) tubers are multi-segmented, bearing 4 to 7 dominant buds. Tuber surfaces are brown (RHS 200C) with the cortical area yellow-orange (RHS 10C).
   Inflorescence.—The inflorescences of ‘UF404’ have been observed only on shadehouse-grown plants. Inflorescence Arrangement: Upright hooded spathes surrounding a columnar spadix borne on a tall upright scape.
   Fragrance.—None detected.
   Natural flowering season/longevity.—Plants of ‘UF404’ typically flower during the spring or early summer in central Florida. Flowers develop about seven weeks after growth commences. Inflorescences last about three days before fading; inflorescences persistent.
   Spathe.—Length: 11.5 cm. Width, distal: 3.5 cm. Width, proximal: 2.2 cm. Shape: Ovate to somewhat obovate. Apex: Acute to acuminate. Base: Tapering. Margin: Entire; proximal, notched. Texture (upper and lower surfaces): Smooth, glabrous. Color: Front Surface: Upper two-thirds: yellow-white (RHS 158D), becoming grey-brown (RHS 199D) with maturity; lower one-third: yellow-green (RHS 144A) to yellow-green (RHS 144C). Rear Surface: Upper two-thirds: yellow-green (RHS 145B); lower one-third: yellow-green (RHS 148D) streaked with yellow-green (RHS 147C).
   Spadix.—Spadix with sessile, simple female and male flowers separated into two zones. Female flowers arranged on the lower one-third of the spadix; male flowers arranged on the upper two-thirds of the spadix. Sterile flowers develop between female and male flower zones. Near this area, the spathe constricts surrounding the female flowers. Length (entire spadex): 8 cm. Length (male flower zone): 4.5 cm. Length (sterile flower zone): 1.8 cm. Length (female flower zone): 1.7 cm. Diameter (male flower zone): 8.5 mm. Diameter (sterile flower zone): 7 mm. Diameter (female flower zone): 8 mm. Shape: Spindle-shaped to columnar. Apex: Obtuse. Base: Obtuse. Aspect: Upright. Color (mature, male zone): yellow-white (RHS 158B). Color (mature, sterile zone): yellow-white (RHS 158B). Color (mature, female zone): yellow (RHS 10C).
   Male flowers.—Quantity per spadix: About 140. Shape: Obovate. Height: 2.5 mm. Diameter: 1.5 mm. Anther color: RHS 157D. Amount of pollen: Moderate.
   Female flowers.—Quantity per spadix: About 150. Shape: Obovate. Height: 2 mm. Diameter: 1.5 mm. Stigma color: RHS 9C. Ovary color: RHS 155D.
   Scape.—Length: 14 cm. Diameter (distal): 5.3 mm. Diameter (proximal): 6.5 mm. Strength: Sturdy, flexible. Aspect: Erect. Texture: Smooth, glabrous. Color: Greyed-Purple (ranging from RHS 187A to RHS 185A).
Seed and fruit: No Fruit or Seed without hand pollination.

### COMPARISON WITH KNOWN CULTIVARS

Plants of ‘UF404’ differ from plants of the female parent, ‘Florida Blizzard’ (U.S. Plant Pat. No. 14,406) in the following characteristics:

1. Plants of ‘UF404’ are much shorter than plants of the female parent;

2. Plants of ‘UF404’ differ from plants of the female parent in leaf shape, as plants of the female parent have fancy (or heart) shaped leaves; and

3. Plants of ‘UF404’ differ from plants of the female parent in leaf coloration, as leaves of the plants of ‘Florida Blizzard’ have white veins and white blotches in a green background.

Plants of ‘UF404’ differ from plants of the male parent, ‘Florida Red Ruffles’ (U.S. Plant Pat. No. 13,136), in the following characteristics.

1. Plants of ‘UF404’ are more vigorous and larger than plants of the male parent;

2. Leaves of the plants of ‘UF404’ are longer and wider than leaves of the plants of the male parent; and

3. Plants of ‘UF404’ and ‘Florida Red Ruffles’ differ in leaf color, as leaves of ‘Florida Red Ruffles’ are dull red in the center and have narrow green margins.

Additionally, comparisons were made with the commercial varieties ‘Red Frill’, ‘Florida Red Ruffles’, and ‘Florida Sweetheart’ (U.S. Plant Pat. No. 8,526). Comparisons for chilling resistance were made with the two commercial varieties ‘Florida Red Ruffles’ and ‘Candidum’. All comparisons were made in Wimauma, Fla. in 2006 and 2007.

Field plots were organized in a randomized complete block design consisting of three replications, and each plot contained 30 propagules. Tubers were dug in December 2006 and January 2008, respectively. Dried tubers were graded by maximum diameter; No. 2 (2.5 to 3.8 cm), No. 1 (3.8 to 6.4 cm), Jumbo (6.4 to 8.9 cm), Mammoth (8.9 to 11.4 cm), and Super Mammoth (>11.4 cm). The production index, an indicator of economic value of the harvested tubers, was calculated as: N (No. 2)+2N (No. 1)+4N (Jumbo)+6N (Mammoth)+8N (Super Mammoth); where N=number of tubers in each grade. In 2006, ‘UF404’ produced similar numbers of marketable tubers (44 to 52) as ‘Florida Red Ruffles’ and ‘Florida Sweetheart’ did, but ‘UF404’ had the greatest tuber weight and the highest production index, with its tuber weight 95% and 68% greater than ‘Florida Red Ruffles’ and ‘Florida Sweetheart’, and production index 56% and 39% higher than ‘Florida Red Ruffles’ and ‘Florida Sweetheart’ (Table 1). ‘UF404’ was superior to ‘Red Frill’ in tuber weight (3.9×), production index (2.3×) and marketable number of tubers (1.3×). In 2007, ‘UF404’ was comparable to ‘Florida Red Ruffles’ and ‘Florida Sweetheart’ in tuber weight, production index, and marketable tubers, and it was superior to ‘Red Frill’ in all these assessments. In tuber size distribution, the majority (60% or more) of tubers for ‘UF404’ were in the categories of No.1, Jumbo, or Mammoth which was similar to ‘Florida Red Ruffles’ and ‘Florida Sweetheart’. ‘UF404’ produced larger tubers than ‘Red Frill’, which had the majority (>90%) of tubers in the No.1 and No.2 categories.

Landscape performance of the varieties grown under full-sun conditions was evaluated in 2006 and 2007 on the same plots used for evaluating tuber production. The overall plant performance was rated multiple times (July, August, and September) in each growing season, on a scale of 1 to 5, with 1 being very poor (few leaves and lack of vigor), and 5 being excellent (full plants, numerous leaves, and bright color display). Similarly, leaf sun burn tolerance was also evaluated multiple times in each growing season on a scale of 1 to 5, with 1 being very susceptible to sun burns and showing numerous sun-damaged areas or holes on leaves and 5 being resistant to sun burns and not showing any sun-damaged areas. At approximately 4 months after planting, plant height, number of leaves, and foliar characteristics were measured on three randomly chosen plants in each plot. All varieties tested produced a similar high number of leaves typical of lance-leaved plants (Table 2). ‘UF404’ plants were taller (5 to 12 cm taller), and its leaves were larger (4 to 10 cm longer and 1 to 6 cm wider) than those of ‘Red Frill’, ‘Florida Red Ruffles’, and ‘Florida Sweetheart’. With the multiple, larger leaves, ‘UF404’ formed full plants with excellent color display, resulting in higher performance ratings than ‘Red Frill’, ‘Florida Red Ruffles’, and ‘Florida Sweetheart’ in most of the comparisons. In sun burn tolerance ratings, ‘UF404’ was similar to ‘Florida Red Ruffles’ and ‘Florida Sweetheart’ (4.0 to 4.4 through the growing season) and was better than ‘Red Frill’ in August and September.

The varieties' suitability for container forcing was evaluated by forcing tubers in 11.4-cm containers. No. 1 tubers were planted either intact or de-eyed in a peat/vermiculite mix on 26 Mar. 2007. The study was conducted in a greenhouse with 45% light exclusion during the summer in Wimauma, Fla. Average daily temperatures ranged from a low of 16° C. night to 29° C. day during the experiment. Potted plants were arranged on metal benches in the greenhouse in a randomized complete block design with 10 replications. Plant height, number of leaves, and foliar characteristics were recorded 8 weeks after planting. ‘UF404’ produces excellent pot plants regardless of tuber treatments (intact or de-eyed) (Table 3). When tubers were planted intact, ‘UF404’ sprouted 30 days after planting, similar to ‘Red Frill’ (32 days), but 9 to 13 days earlier than ‘Florida Red Ruffles’ and ‘Florida Sweetheart’. When tubers were de-eyed, ‘UF404’ still sprouted earlier than ‘Florida Red Ruffles’ and ‘Florida Sweetheart’. ‘Red Frill’ sprouted ˜6 days earlier than ‘UF404’ when tubers were de-eyed. Early sprouting is preferred by greenhouse growers as it allows for a quicker crop cycle.

Intact or de-eyed plants of ‘UF404’, ‘Florida Red Ruffles’ and ‘Red Frill’ were similar in height and a little taller than those of ‘Florida Sweetheart’. Although it produced similar numbers of leaves as the comparison varieties, ‘UF404’ had leaves 4 to 6 cm wider than ‘Florida Sweetheart’ and ‘Red Frill’. ‘UF404’ and ‘Florida Red Ruffles’ were not significantly different in leaf width at the eighth week after planting. With multiple, bright colored leaves, containerized ‘UF404’ plants received the highest (4.5 to 4.9) quality ratings when compared to ‘Red Frill, ‘Florida Red Ruffles’, and ‘Florida Sweetheart’ (3.0 to 4.3).

‘UF404’ was also evaluated for its resistance to chilling injuries. Mature leaves were excised from approximately 3-month-old potted plants and exposed to 7.2° C. for 3 days in a growth chamber. The leaves were then held at ambient temperature for 10-11 days before examined for signs of chilling injuries (chlorotic and necrotic areas) and assessed for percentage of leaf area injured. Four chilling tests were conducted in June to August 2007, with four replicates in each test. ‘Florida Red Ruffles’ and ‘Candidum’ (unpatented) were used as the chilling-resistant and chilling-susceptible controls, respectively. Leaves of ‘UF404’ had less areas injured by the chilling treatment than both ‘Candidum’ and ‘Florida Red Ruffles’, indicating ‘UF404’ has a good level of resistance to chilling injuries (Table 4).

TABLE 1
Tuber weight, production index, number, and tuber grade distribution of
‘UF404’ and three commercial cultivars harvested in 2006 and 2007.
Values presented are means of three replications with 30 propagules
planted in a plot per year.

| | Tuber | | |
|---|---|---|---|
| | Weight | Production | Marketable |
| Cultivar | (kg) | index | (no.) |
| | Year 2006 | | |
| UF404 | 4.7 | 155 | 52 |
| Red Frill | 1.2 | 68 | 39 |
| Florida Red Ruffles | 2.4 | 99 | 44 |
| Florida Sweetheart | 2.8 | 111 | 50 |
| | Year 2007 | | |
| UF404 | 2.8 | 105 | 37 |
| Red Frill | 1.9 | 66 | 39 |
| Florida Red Ruffles | 2.7 | 84 | 42 |
| Florida Sweetheart | 3.3 | 84 | 46 |

| | Distribution of marketable tubers (%) | | | | |
|---|---|---|---|---|---|
| | Super | | | | |
| Cultivar | mammoth | Mammoth | Jumbo | No. 1 | No. 2 |
| | Year 2006 | | | | |
| UF404 | 1 | 11 | 31 | 47 | 11 |
| Red Frill | 0 | 0 | 1 | 67 | 32 |
| Florida Red Ruffles | 0 | 7 | 21 | 30 | 43 |
| Florida Sweetheart | 1 | 3 | 20 | 49 | 27 |
| | Year 2007 | | | | |
| UF404 | 0 | 3 | 21 | 51 | 25 |
| Red Frill | 0 | 0 | 8 | 45 | 47 |
| Florida Red Ruffles | 0 | 1 | 20 | 41 | 38 |
| Florida Sweetheart | 0 | 3 | 29 | 31 | 38 |

The production index is an indicator of economic value of the crop harvested and is calculated as: N (No. 2s) + 2N (No. 1s) + 4N (Jumbos) + 6N (Mammoth) + 8N (Super Mammoth); where N = number of tubers in each grade.
Tubers graded by maximum diameter; No. 2 (2.5 to 3.8 cm), No. 1 (3.8 to 6.4 cm), Jumbo (6.4 to 8.9 cm), Mammoth (8.9 to 11.4 cm), and Super Mammoth (>11.4 cm).

TABLE 2
Plant characteristics, performance, and sun tolerance ratings of ‘UF404’
and three commercial cultivars grown from planting 2.54-cm caladium
tuber propagules in ground beds in full sun (2006 and 2007). Values
presented for plant height, leaf number, length and width are means of
three replications with three plants measured per plot per year, while
performance and sun bum tolerance ratings are means of three replications
based on whole plot evaluation.

| | plant | | Leaf | Leaf |
|---|---|---|---|---|
| | height^{z} | Leaves^{z} | length^{z} | width^{z} |
| Cultivar | (cm) | (no.) | (cm) | (cm) |
| UF404 | 27 | 25 | 24 | 14 |
| Red Frill | 14 | 21 | 15 | 8 |
| Florida Red Ruffles | 19 | 23 | 18 | 11 |
| Florida Sweetheart | 21 | 22 | 20 | 13 |

| | Performance rating^{y} | | | Sun tolerance rating^{x} | | |
|---|---|---|---|---|---|---|
| | | | Septem- | | | Septem- |
| Cultivar | July | August | ber | July | August | ber |
| UF404 | 4.0 | 3.5 | 4.3 | 4.0 | 4.4 | 4.4 |
| Red Frill | 4.2 | 2.6 | 2.3 | 4.3 | 3.8 | 3.3 |
| Florida Red Ruffles | 3.5 | 2.5 | 3.0 | 4.3 | 4.2 | 4.4 |
| Florida Sweetheart | 3.5 | 3.2 | 3.4 | 4.2 | 4.1 | 4.2 |

^{z}Data were taken in August 2006 and 2007, approximately four months after tubers were planted in April each year, and averaged of two growing seasons (2006 and 2007).
^{y}Plants were rated on a scale of 1 to 5, with 1 being very poor, 3 fair and acceptable, and 5 being excellent in plant vigor, fullness, and color display.
^{x}Plants' sun burn tolerance was rated on a scale of 1 to 5, with 1 being very poor, 3 fair and acceptable, and 5 being excellent without showing any signs of leaf burns or resulted holes on leaf surfaces.

TABLE 3
Plant performance for caladium cultivars grown from No. 1 tubers in 11.4-
cm containers in a 45% shaded glasshouse, 2007, Wimauma, Fla. Values
represent the means of 10 plants produced from intact or de-eyed No. 1
(>3.8 and <6.4 cm in diameter) tubers planted individually per container.
Data was taken 8 weeks after planting.

| | Days to | | Plant height | | | |
|---|---|---|---|---|---|---|
| | sprout^{z} | | (cm) | | Leaves (no.) | |
| Cultivar | Intact | De-eye | Intact | De-eye | Intact | De-eye |
| UF404 | 30 | 38 | 18 | 16 | 10 | 12 |
| Red Frill | 32 | 32 | 17 | 16 | 10 | 18 |
| Florida Red Ruffles | 38 | 40 | 16 | 17 | 8 | 13 |
| Florida Sweetheart | 43 | 39 | 10 | 12 | 6 | 16 |
| | Leaf length | | Leaf width | | | |
| | (cm) | | (cm) | | Quality rating | |
| Cultivar | Intact | De-eye | Intact | De-eye | Intact | De-eye |
| UF404 | 25 | 20 | 17 | 14 | 4.5 | 4.9 |
| Red Frill | 18 | 16 | 11 | 10 | 3.2 | 3.9 |
| Florida Red Ruffles | 17 | 18 | 13 | 12 | 3.3 | 4.3 |
| Florida Sweetheart | 15 | 16 | 11 | 11 | 3.0 | 4.2 |

^{z}Number of days from planting to the first unfurled leaf.

TABLE 4
Sensitivity of ‘UF404’ and two commercial varieties to
chilling injuries caused by exposing to 7.2° C. for 3 days.

| Cultivar | Leaf area injured (%)^{y} | Leaf injury score^{z} |
|---|---|---|
| UF404 | 43 | 7.4 |
| Candidum | 90 | 9.8 |
| Florida Red Ruffles | 63 | 8.5 |

^{y}Percentage of leaf area injured was assessed 10-11 days after chilling.
^{z}Scores were made on a scale of 1 to 10: 1 = no injury, bright and healthy as control's, 2 = some discoloration or yellowing, 3 = 1 to 2%, 4 = 3 to 5%, 5 = 6 to 10%, 6 = 11 to 20%, 7 = 21 to 35%, 8 = 36 to 55%, 9 = 56 to 75%, and 10 = 76 to 100% of the total leaf area injured.

I claim:

1. A new and distinct cultivar of Caladium plant as shown and described herein.
